(19) United States
(12) Patent Application Publication
(10) Pub. No.: US 2006/0115888 A1
(43) Pub. Date: Jun. 1, 2006

(54) Large-scale electroporation plates, systems and methods of use

(75) Inventors: Andre S. Gamelin, Vista, CA (US); Daniel D. Holt, San Diego, CA (US); Christian S. Andre, San Diego, CA (US)

Correspondence Address: BIOTECHNOLOGY LAW GROUP;C/O PORTFOLIOIP, PO BOX 52050, MINNEAPOLIS, MN, 55402, US

(73) Assignee: Genetronics, Inc., San Diego, CA (US)

(21) Appl. No.: 10/537,203

(22) PCT Filed: Dec. 3, 2003
(86) PCT No.: PCT/US03/38470

§ 371 Date: Jun. 3, 2005

Related U.S. Application Data
(60) Provisional application No. 60/430,738, filed on Dec. 3, 2002.

(51) Int. Cl. C12M 1/42

(52) U.S. Cl. 435/285.2; 435/288.4; 435/305.1

(57) ABSTRACT

Described are electroporation plates (2) that comprise a plurality of energizable electroporation wells (4) or chambers arrayed in a solid substrate, wherein at least two of the wells (4) in the plate (2) can be independently energized. Each well (4) contains at least two electroporation electrodes (12, 14) disposed therein, and serves as the vessel in which an individual electroporation reaction can be conducted. Also, described are electroporation systems that use electroporation plates (2) according to the invention, as well as methods of using such electroporation plates (2) and systems, for example, to optimize electroporation conditions.

### RELATED APPLICATIONS

This application claims priority to and the benefit of U.S. provisional patent application Ser. No. 60/430,738, filed 3 Dec. 2002, entitled “Multiple Well Electroporation Plates”, under 35 U.S.C. §119(e).

### TECHNICAL FIELD

This invention relates to electroporation. More particularly, the invention concerns large-scale (e.g., large volume, multiple well, etc.) electroporation plates, systems for use in conjunction with such plates, and methods relating to the use of such plates.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

Electroporation is a well-established technique for moving exogenous molecules, including nucleic acids, drugs, and other compounds, across membranes, including cell membranes and the membranes that form liposomes and other lipid-encapsulated vesicles. It involves the application of electric fields of suitable strength across a sample containing, for instance, cells to be made competent for the introduction of molecules of interest. While the mechanism by which electroporation functions is not fully understood, in the context of living tissue electroporation is known to involve the breakdown of the cell membrane's lipid bilayer, leading to the formation of transient or permanent pores in the membrane that permit exogenous molecules to enter the cell by diffusion.

In the context of ex vivo methods for introducing exogenous molecules into cells and other lipid-enclosed vesicles, electroporation offers numerous advantages: it can be used to treat whole populations of cells (or vesicles) simultaneously; it can be used to introduce essentially any macromolecule into a cell (or vesicle); it can be used with a wide variety of primary or established cell lines and is particularly effective with certain cell lines; and it can be used on both prokaryotic and eukaryotic cells without major modifications or adaptations to cell type and origin. Additionally, electroporation can be used on cells in suspension or in culture, as well as cells in tissues and organs. Cells can also be electroporated prior to exposure to the molecular species to be introduced into the cell. Indeed, cells can be rendered highly competent for transfection or transformation by electroporation, after which they can be stored prior to exposure to the molecules (e.g., expression vectors encoding one or more genes of interest) to be introduced.

Typically, ex vivo electroporation is conducted by positioning a single-channel apparatus that includes at least a pair of electrodes, i.e., a cathode and an anode, in a sample-containing chamber such as a disposable cuvette. See, e.g., Neumann et al., Biophysical Journal, 71, pp. 868-77 (1996). An electric potential is typically applied using a generator that emits pulses of a high-voltage electric field to a solution or suspension containing a cell population obtained from a patient, and whether the pore formation is reversible or irreversible depends on such energizing parameters as pulse amplitude, duration, wave form shape, and repetition rate, in addition to the type and development stage of the cells. It is believed that pore formation in, or permeabilization of, the membrane occurs at the cell poles, the sites on the cell membranes that directly face the electrodes and thus experience the highest transmembrane electric field potential. Unfortunately, in a given electroporation experiment, the degree of permeabilization may vary with the cell type, and even among cells in a given population. Variation is also frequently seen from experiment to experiment using the same sample electrolyte conditions and cell type. Furthermore, since the procedure may be performed in large populations of cells whose individual properties vary, electroporation conditions typically can only be selected to address the “average” qualities of the particular cell population.

As mentioned above, electroporation has traditionally been conducted in disposable single chamber cuvettes, which typically have a maximal volume of about one milliliter (mL) for purposes of electroporation. Such techniques, however, are tedious, labor intensive, and require optimization. To date, efforts to increase the throughput of electroporation processes have revolved around multi-channel electrode systems, which have been used for quasi-high throughput introduction of exogenous molecules into cells in an attempt to limit the need for transferring cells from culture containers to electroporation cuvettes. A conventional multi-channel electroporation apparatus includes a plurality of pairs of electrodes that can be inserted in respective ones of a plurality of chambers that hold the exogenous materials and the cells. Currently available multi-channel electroporation devices contain 8 or 96 pairs of coaxial electrodes (Genetronics, Inc., San Diego, Calif.). These devices are used for electroporation in standard 96-well plates, which consist of 8 rows and 12 columns of wells and have a standard size of about 8.5 (W) centimeters (cm) by about 12.7 cm (L), with a standard center-to-center spacing of approximately 9.0 millimeters (mm) between wells. See U.S. Pat. No. 6,352,853.

While conventional quasi-high throughput electroporation devices for electroporation of multiple cell samples or populations have found limited application, they possess inherent flaws that limit their widespread adoption. These flaws include that such devices employ two separate components, namely a multi-well plate and an electrode array that is inserted into the wells after the various reagents (e.g., a suspension of containing host cells in a suitable buffer and the exogenous molecules to be electroporated into the cells) have been added. In such currently available systems, while the multi-well plates may be disposable, the electrode array is not, and must be cleaned after each use, thereby greatly limiting the true throughput and automation of such systems. Another major flaw in the design of such systems is that the electrodes of such arrays, after introduction into the wells, cannot be optimally positioned in the wells, as some clearance space must be left inserting the electrodes into the wells. As such clearance space reduces the percentage of the sample exposed to the electric field, electroporation efficiency is necessarily reduced. Also, most electrode arrays for such systems employ multiple cylindrical outer electrodes each positioned about a central pin electrode. Such a configuration inherently results in electric fields of varying strengths at different locations in each well. Another disadvantage of such systems is that only a single set of energizing parameters can be investigated on any one plate, as the electrode pairs in the arrays typically used in such systems are not capable of being energized independently from one or more of the other electrode pairs in the array. Thus, optimization experiments require the use of at least one multi-welled plate for each set of energizing parameters selected. Other flaws are also known to those familiar with such devices, including the loss of sample due to wicking of the sample onto the electrodes.

In addition to the above shortcomings, when large volumes are to be electroporated, conventional techniques typically rely on “flow through” systems, where a portion of the total volume to be electroporated is moved into an electroporation chamber and the electroporating pulses are applied. The chamber is then emptied and refilled as many times as necessary to electroporate all of the cells in the total volume of cells to be electroporated. The reasons for such repetition of pulsing are several fold. For example, conventional, high voltage electroporation pulse generators can typically deliver energy to only about four 1 mL cuvettes at a given time. An additional drawback is that the requirement for multiple pulsing on the sample is deleterious to the cells in the sample often resulting in unacceptable death rates of the cells in certain applications where the incidence of molecular transfer into the cells of the sample is known to be low. See, e.g., U.S. Pat. Nos. 6,207,488, 5,676,646, and 5,545,130 for descriptions of flow-through electroporation devices and their use.

Given the limitations of conventional electroporation devices as they relate to throughput and volume, the need clearly exists for devices and systems suitable for high throughput and other large-scale applications. The instant invention addresses these and other needs in the electroporation arts, as described below.

Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the term “contacting” refers to any method of exposing a host cell to an exogenous molecule. For example, a host cell, or population of host cells, can be immersed or bathed in an electroporation buffer solution containing one or more species of exogenous molecules. Contact between a host cell and an exogenous molecule may occur either before or after application of electric impulse(s) via electroporation. In response to such contact, an exogenous molecule is “introduced” into a host cell when the exogenous molecule enters the host cell and exhibits either a transient or stable effect, for example, expression of a heterologous nucleic acid molecule in the host cell (e.g., the production of biologically green fluorescent protein encoded by an expression vector).

“Energizing parameters” refer to the particular energizing characteristics delivered by a pair of electroporation electrodes in a given experiment. Such parameters include pulse amplitude, duration, wave form shape, repetition rate, and interval between pulses.

An “exogenous molecule” refers to any molecule intended for introduction into to a host cell. Exogenous molecules include nucleic acid molecules (e.g., expression vectors carrying one or more genes or heterologous nucleic acids encoding proteins or polypeptides to be expressed, anti-sense molecules, ribozymes, small interfering RNAs, etc.), small molecule drugs, and other chemicals that somehow alter, transiently or permanently, the cell or its function(s), including the biochemical activity of any protein or enzyme in the cell.

The term “genetically modified” refers to the introduction of one or more heterologous nucleic acids into one or more host cells. A “heterologous nucleic acid” refers to a nucleic acid molecule that originates from a foreign species, or, if from the same species, is substantially modified from its original form or will result in an increase in the copy number of the introduced nucleic acid.

A “host cell” is any cell into which an exogenous molecule can be introduced. Host cells include eukaryotic and prokaryotic cells. Eukaryotic host cells include animal, plant, and fungal cells. Preferred animal cells include those from mammals (e.g., bovine, canine, equine, feline, murine, ovine, and porcine animals), including humans and primates, fish (e.g., zebrafish, salmon, trout, and other commercially important species), insects (e.g., the fruit fly, mosquitoes, bees), arachnids, birds, crustaceans, and mollusks, as well as cell lines derived from cells of any of the foregoing organisms. Preferred plant cells include those from crop plants such as cereal grains, as well as ornamental plants and trees grown for lumber production. Preferred fungal cells are yeast cells. Preferred prokaryotic cells are bacterial cells, particularly species useful in molecular biology. Host cells into which a heterologous nucleic acid is introduced are referred to as “recombinant host cells”. Host cells also include artificial cells, liposomes, and any other lipid-encapsulated vesicle into which an exogenous molecule can be introduced by electroporation. For purposes of brevity, “host cells” may also be referred to herein simply as “cells”.

Performing a method “in vitro” refers to performing the method outside of an organism, and includes the concept of ex vivo methods, wherein, for example, a sample of cells is removed from a patient and electroporated using the devices and methods of the invention to introduce one or more species of exogenous molecules into the cells, after which the treated cells are reintroduced into the patient.

“Large-scale” means either or both that the electroporation plates of the invention are amenable to high throughput and/or large volume applications. Here, “high throughput” refers to the ability to perform two or more, preferably, 4, 16, 32, 64, 96, 192, 288, 384, 576, 768, 672, 1536, 3072, or 6144 or more identical or different electroporation experiments on the same electroporation plate.

A “large volume” application refers to a volume that exceeds the volume of a conventional electroporation cuvette or other vessel for conducting a single electroporation experiment. Typically, conventional electroporation cuvettes or other vessels provide of volume of up to about 1 mL, although vessels that may contain up to about 10 mL are known, particularly in the context of flow-through electroporation devices. In the context of this invention, the vessel may contain a volume from as small as about 1 microliter (uL) to about 10 mL or more. Preferred large volume applications allow for the electroporation of cell-containing solutions having a volume of at least about 5 mL, preferably at least 10 mL to about 100 mL or more.

As used herein, the term “nucleic acid” or “nucleic acid molecule” refers to a polymer of deoxyribonucleotides or ribonucleotides (in either case, a “polynucleotide”) in the form of a separate fragment or as a component of a larger construct. Nucleic acids may be either single- or double-stranded, and include non-naturally occurring bases or backbone structures. Such molecules include DNA, RNA, cDNA, antisense, ribozyme, and triplex forming molecules. Nucleic acids can be naturally occurring or synthetic, and include oligonucleotides. DNA encoding proteins or polypeptides utilized in the methods of the invention can be assembled, for example, from cDNA fragments or from oligonucleotides that provide a synthetic gene that is capable of being expressed in a recombinant transcriptional unit. Polynucleotide or nucleic acid sequences of the invention include DNA, RNA, and cDNA sequences.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application issues as a patent to a time the validity of one or more of the appended claims is questioned, the claims are to be interpreted in a way that (1) preserves their validity and (2) provides the broadest reasonable interpretation under the circumstances.

“Plant” refers either to any whole plant, a plant part, a plant cell, or a group of plant cells, such as plant tissue, for example. The classes of plants whose cells can be used in conjunction with the electroporation plates of the invention include any higher plant, be they monocotyledonous or dicotyledonous plants, and any ploidy level, including polyploid, diploid, and haploid. “Monocotyledonous plants”, or “monocots”, include asparagus, field and sweet corn, barley, wheat, rice, sorghum, onion, pearl millet, rye and oats. Examples of “dicotyledonous plants”, or “dicots”, include tomato, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers, and various ornamentals.

“Plant cell” refers to an intact cell of any plant, including a cell from a leaf, callus, embryo, or seed, as well as any gamete-producing cell and any cell that regenerates into a whole plant. Thus, a seed comprising multiple plant cells capable of regeneration into a whole plant is included in the definition of a plant cell. In this context, term “intact” refers to a single cell or group of single cells which form a tissue, wherein the cell(s) have undamaged or untreated cell wall(s), as compared to protoplasts.

A “plurality” means more than one.

### SUMMARY OF THE INVENTION

It is an object of this invention to provide large-scale electroporation plates, and systems and kits employing such plates, to overcome the deficiencies of current approaches for introducing exogenous molecules into cells, particularly in vitro. Another object of the invention is to provide methods of using such electroporation plates, for example, to introduce an exogenous molecule into a host cell in vitro.

Thus, one aspect of the invention concerns electroporation plates that comprise a plurality of energizable electroporation wells arrayed in a solid substrate, wherein at least two of the wells in the plate can be independently energized, i.e., when energy is provided to the electroporation electrodes in one of the two wells, the electrodes in the other well need not be energized, although they may be. Each well contains at least two electroporation electrodes (at least one anode and at least one cathode) disposed therein, and serves as the vessel in which an individual electroporation reaction can be conducted.

In preferred embodiments of this aspect, the wells of an electroporation plate are arrayed in a series of at least two rows and two columns. In particularly preferred embodiments, the number of rows differs from the number of columns. Typically, but not essentially, the ratio of rows to columns is about 2:3. In some preferred embodiments, the plates of the invention comprise 96 electroporation wells arrayed in 8 rows and 12 columns. In other preferred embodiments, the plates comprise 384 electroporation wells arrayed in 16 rows and 24 columns. Other preferred plate configurations contain 192, 288, 576, 672, 768, 1536, 3072, or 6144 wells. To facilitate automated handling and compatibility with existing plate-based high throughput systems used in the biological sciences, it is preferred that the overall dimensions of the plates (length, width, and height) be the same plate to plate, regardless of the number of rows, columns, and electroporation wells, as compared to conventional, non-electrifiable multi-well plates (e.g., as are currently used in automated high throughput compound screening systems).

In many embodiments, particularly in plates having fewer than about 384 electroporation wells, the wells are substantially cylindrical or rectangular boxes, in either case with one end, at the top of the plate, being open. In other embodiments, especially in those having large numbers of electroporation wells, the wells tend to be substantially rectangular boxes with one open end (at the top), such that when viewed from the top, their length and width dimensions are substantially similar (yielding a square shape), with the depth dimension being different, typically larger, than the length and/or width dimension. Of course, wells having any suitable geometric shape may be employed in the context of the invention, although preferred well configurations provide at least two spaced, substantially parallel walls upon which one or more electrode pairs are disposed. In any event, it is preferred that the electroporation wells of a plate have substantially uniform dimensions when compared well-to-well. Such consistency in terms of shape serves to provide a substantially uniform electroporation response from well to well, all else being equal. As electroporation wells are typically open at the top (absent a removable cover plate, seal, or the like), each well has only a bottom wall and at least one sidewall (one in the case of a cylindrically shaped well, four in the case of a rectangle- or square-shaped well, etc.). As will be appreciated, to facilitate manufacture, for example, by injection molding techniques, when viewed from the side, a well tends to be slightly tapered, with the upper opening having dimensions (i.e., lengths and widths, or diameters) slightly larger than those at the bottom of the well, thereby facilitating removal of the plate from a mold.

As will be appreciated, for plates having standardized outer dimensions, increasing the number of electroporation wells typically results in a decrease in individual well volume. In some embodiments of the invention, an electroporation plate will contain relatively few wells (e.g., 96 or even as few as 2-12), enabling each well to have volume of about 1 milliliter (ml), in the case of a 96 well plate, and 10 ml in the case of 12 wells. Preferred well volumes range from about 10 mL to down to about 1 microliter (uL), although smaller volumes are possible, particularly if, for example, photolithographic techniques from the semiconductor fabrication industry are adapted to generate plates containing electroporation wells having volumes on the microliter, and even nanoliter, scale.

Preferably, in each electroporation well (or chamber) of an electroporation plate according to the invention, one of the electroporation electrodes for that well will be positioned, or disposed, opposite to the other electroporation electrode for that well. Preferably, the electrodes in a given well are substantially parallel to each other to facilitate generation of a uniform electric field in the well when its electrodes are energized in the presence of an electrically conductive solution. In this context, substantially parallel means +/−10%, preferably 5%, and even more preferably, 1% or less. Electrodes may be disposed in the wells in any suitable direction, although preferably the electrodes are horizontally or vertically disposed, as viewed when the plate is placed on a surface to receive a sample of cells. Electrodes may, in any given well in, for example, large volume wells, also comprise a plurality of individual electrodes (preferably arrayed in pairs of opposed electrodes, one being an anode, the other being a cathode) that may be energized together or in succession and in a particular pattern facilitating the substantially uniform distribution of electric fields through out the well.

Electroporation electrodes are preferably integrated into the sidewall(s) of a well in a fluidly sealed manner (to prevent leakage of liquid after it is added to the well) such that a conducting surface thereof is exposed to a sample placed in the well. Alternatively, to form an electrode, electrode material (e.g., gold) can be deposited, preferably as a thin film, or layer or composite of thin films, on a sidewall of the well. Processes for deposition of electrode material include dipping, plating (electroless or electrolytic), spraying, and vapor deposition. Electrodes (or portions thereof to be exposed to host cells) are preferably made of biocompatible, electrically conductive materials. Examples of such materials include gold, aluminum, titanium, or a non-metallic, electrically conductive composite such as graphite (or a polymer filled or sufficiently doped with an amount conductive material sufficient for the composition to serve as an electrode useful in the practice of the invention).

In certain embodiments, the electrodes comprise a composite of multiple electrode materials, e.g., copper, nickel, and gold, each of which is applied to the solid substrate at different times, thereby building up layers of electrode material. Preferably, in such composites, the outermost layer of electrically conductive material (i.e., that to be exposed to cells) is biocompatible. The electrode material(s) in the layer(s) below the outermost layer may also be biocompatible, although factors such as, for example, cost, compatibility with the underlying substrate or adhesion layer, if any, ease of manufacture or application, and the material's properties as a conductor may influence the skilled artisan's material selection for a given application.

When applied to a sidewall, an electrode preferably covers as much of the sidewall as is required to generate a substantially uniform electric field in the well (or volume between the electrodes) upon being energized in conjunction with its complementary electrode(s). As will be appreciated, in large-scale applications where a plurality of electrode sets (e.g., pairs) that are energized at different times are employed, the substantially uniform electric field is generated in the volume of cell-containing solution between the electrodes that comprise the particular electrode set being energized, not necessarily in the entire chamber. Preferably the electrodes cover as much of the sidewalls as possible, as this allows a wide range of solution volumes, up to the maximum for the given well, to be used, in addition to allowing the uniformity of the electric field generated between the electrodes to be maximized.

Each electrode in an electroporation plate is connected to a conductor (e.g., an electrically conductive wire or contact point) that can carry energy to the particular electrode from a power source (e.g., a pulse generator) that can be connected to the device. In certain preferred embodiments, the electrodes for the electroporation wells in a given column (or row) are interconnected in series or in parallel such that they can be energized, or addressed, simultaneously, while the electrodes for the electroporation wells in other columns (or rows) of the plate not electrically connected to the energized wells will not be energized. In other preferred embodiments, the electrodes for each well of an electroporation plate can be independently addressed, thus allowing each well of the plate to be energized independently of all of the other wells in the plate. Of course, in a plate having independently addressable wells, it may be desirable to similarly energize two or more wells, for example, to generate statistics related to the transformation efficiency of the particular energizing parameters.

The electroporation plates of the invention are made from any suitable solid substrate. In certain embodiment, the solid substrate is made from a homogenous composition of a single material, while in certain other embodiments, it can be made from combinations of different materials. Particularly preferred substrates are plastics, since plastics are inexpensive, easily formed into desired shapes (for example, by various molding processes, such as injection molding), resistant to breakage, have desirable optical properties, and are easily machined, if desired. In preferred embodiments, all or a portion of the solid substrate will be transparent to light of one or more wavelengths (e.g., wavelengths in the visible electromagnetic spectrum). In other embodiments, all or a portion of the solid substrate will be translucent, while in still other embodiments, all or a portion of the solid substrate will be opaque.

Electroporation plates according to the invention also include at least one electrical connection that allows energy to be delivered to the electrodes in the plate. Any suitable electrical connection can be used, including electrical connections that employ pins and sockets. In particularly preferred embodiments, a plurality of contact pad-type electrical connections is provided, with each electrode (or series of electrodes to be energized with the same parameters) being connected to a conductor energized through a separate contact pad. Thus, each contact pad is electrically connected with at least one electrode, thereby allowing the electrode(s) to be energized, if and as desired. The exact number of contact pad connectors depends on the number of electroporation wells (or complete or partial rows or columns of wells) that are to be independently energized, as well as whether other electronic controls (e.g., switches) are provided in a particular circuit.

The electrical connection(s) on an electroporation plate allow energy to be supplied to the one or more electrodes present in the plate, as directed by a controller that directs which, if any, of the electrodes in the plate are to receive energy from the power supply (e.g., a pulse generator). The controller can be built into the power supply or, alternatively, it can be a separate device.

Electroporation plates according to the invention may also contain a tracking or inventory element, for example, a series of optically readable bar codes that provide a unique identifier, so that a particular plate can be tracked in an automated or semi-automated system.

A related aspect of the invention concerns electroporation plates configured to receive a large volume of a cell suspension to be electroporated. In embodiments of this aspect, the plate typically comprises one to about 50, preferably 1 to fewer than about 12, separate chambers. Each chamber may be open at the top, although this not essential. The chamber(s) may also include one or more ports to allow liquid to flow into and/or out of each chamber. Each chamber may also contain one or more internal baffles to limit or control fluid movement within the chamber. In some embodiments, electrodes can be positioned on the baffles. In any event, each chamber comprises at least one, and preferably a plurality of electrode sets disposed on the walls of the chamber. The electrodes comprising each electrode set are disposed on opposite sides of the chamber. Each electrode set is comprised of a plurality of electrodes, preferably an even number of electrodes (e.g., 2, 4, 6, 8, 10, 12, or more) wherein there is an equal number of anodes and cathodes, although the invention contemplates electrodes sets that comprise an odd number of electrodes (e.g., 1, 3, 5, 7, 9, 11, 13, or more). Indeed an electrode set may comprise as few as one electrode, in which event the electrode will be used in conjunction with another electrode set configured accordingly. Particularly preferred are electrode sets that comprise a pair of electrodes (e.g., one anode and one cathode). Each electrode set may be energized independently of the other electrode sets for a given chamber. Similarly, in embodiments that comprise multiple chambers on a single plate, an electrode set in two or more of the chambers may be energized at the same time or at different times.

During electroporation in a particular chamber that comprises a plurality of electrode sets, it is preferred that the electrode sets be energized sequentially. In those embodiments where the electroporation comprises a series of pulses between electrodes in each electrode set, it is preferred that the series of pulses applied between the electrodes of a given electrode set be completed before the electrodes of another electrode set in the chamber are energized, preferably using the same parameters as were used to energize the preceding set(s) of electrodes. By “sequentially” in this context, it is contemplated that, for example, adjacent opposed pairs of electrodes within an elongate well will be “sequentially” energized from one end of an elongated well to the other. Alternatively, the pulsing may be from the center portion of the well to the outer ends of the well.

Another related aspect of the invention concerns kits comprising an electroporation plate according to the invention wherein at least one, some, or preferably, all, of the wells of the plate contain an aliquot of electrically competent host cells. “Electrically competent” refers to host cells that readily uptake exogenous molecules in the presence, or following the application, of a suitable electric field. Typically, the cells will be suspended in a buffer suitable for storage and transport and in which the cells will remain viable for at least 24 hours, preferably 1-3 days, and even more preferably at least 4-7 days. As will be appreciated, different cells may require buffers containing different components. Moreover, it may be desirable to ship cells in a frozen state, in which event a different buffer may again be required. Accordingly, the selection of buffer and cell combinations is left to the artisan's discretion. In order to ship cell-containing plates, a suitable plate cover (e.g., a removable foil or plastic cover) is preferably also provided. To prevent loss of a cell suspension (in liquid or frozen form, as the case may be) from individual wells, it is preferred that the cover individually seal each well. Cell-containing plates may be packaged individually or in bundles of two or more cell-containing plates. A package insert will typically also be provided in each package.

Another aspect of the invention concerns electroporation systems that employ an electroporation plate according to the invention. At a minimum, such systems will comprise at least one such electroporation plate and a power supply adapted for connection to the electrical connector(s) of the electroporation plate. The power supply will also include a controller or, if not, the system will include a controller as a separate device. The controller directs energizing of the plate's various electrodes, preferably according to a preset program, which may be user-defined or factory-provided (for example, selected from a menu listing a plurality of pre-programmed sets of energizing parameters). Such a system may optionally include a plate handler configured to hold the electroporation plate during operation of the electroporation system. In preferred embodiments, the plate handler comprises an electrical connection system compatible with the set of electrodes and conductors in the particular plate such that when a plate is placed therein, the appropriate electrical connections are made, thereby allowing the desired energizing parameters to be delivered to the electrodes in the independently addressable wells in the plate, or in the case of a large-volume plate, to each of the electrode groupings (e.g., opposed pairs of electrodes) that are to be simultaneously energized (e.g. an opposed pairs set). As will be appreciated, one or more of the power supply, controller, and plate handler may be integrated into a single device, or into a collection of devices that number fewer than the various functions provided by a stand alone power supply, controller, or plate handler.

In other embodiments of this aspect, the system further includes one or more plate readers to collect data from the electroporation wells or chamber(s) of the plate. In high throughput systems that use multi-well plates, the reader(s) is(are) typically in a different location. For instance, after electroporation occurs, for example, in a plate handler, the plate may be moved (e.g., by a robot or other device) to a station containing the reader. Data collection then occurs. In some embodiments, such a system may also include an incubation station, where the plate (and the electroporated cells) is incubated under desired conditions prior to being moved to a reading (or data collection) station.

To collect data, a plate reader employs a reading device compatible with the data that will be generated from the electroporation experiment(s) performed in an electroporation plate. For example, if the exogenous molecule introduced into a host cell is a nucleic acid molecule encoding a reporter gene whose expression product is a protein capable of generating fluorescence, a luminomitor may be used to collect data on how much luminescence was emitted from a particular well. Similarly, other species of exogenous molecules may be labeled, as desired, typically with a moiety compatible with the detection system employed. If different electroporation conditions, buffers, reactant concentrations, etc. are used in different wells, different results could be expected. Other reading devices include spectrophotometers (for example, to measure differences in absorbance of a specific wavelength, or range of specific wavelengths, of light in a suspension of host cells into which an exogenous molecule was introduced) and machine vision devices (for example, to assess cell adherence following electroporation-mediated introduction of an exogenous molecule into the host cells in the suspension), although any detection device capable of detecting an effect on cells that is mediated by an exogenous molecule may be employed.

In those embodiments employing a plate reader, it is preferred that the electroporation system also include a data storage device to store data collected by the plate reader for later review and analysis. Any suitable data storage device can be used.

Optionally, an electroporation system will also include a plate storage facility to store electroporation plates during the course or following completion of an electroporation experiment, preferably after a plate reader has collected data.

In other embodiments of this aspect, an electroporation system further comprises an optimization computer adapted to optimize electroporation conditions, alone or in conjunction with other experimental conditions, using electroporation data stored in the memory.

Yet another aspect of the invention relates to methods of introducing an exogenous molecule into a host cell, comprising using electroporation to introduce the exogenous molecule into a host cell present in a suspension contained in an electroporation well of an electroporation plate according to invention. Here, the use of electroporation to “introduce an exogenous molecule into a host cell” refers to rendering the host cell capable of taking up the exogenous molecule using electroporation. As a result of electroporation, the exogenous molecule is then able to diffuse into the host cell. Of course, techniques such iontophoresis can also be employed in order to further enhance the uptake of exogenous molecules into cells which have been electroporated. In certain embodiments, the exogenous molecule is a nucleic acid molecule. In other embodiments, it is a small molecule drug. It will be appreciated that more than one species of exogenous molecule may be present in a given electroporation reaction.

In the instant methods, the host cell typically is a eukaryotic cell or a prokaryotic cell. Preferred eukaryotic host cells include animal cells and plant cells. Particularly preferred animal cells include mammalian cells (e.g., human and primate cells, as well as cells from bovine, canine, equine, feline, murine, ovine, and porcine animals), insect cells, fish cells, bird cells, arachnid cells, mollusk cells, and crustacean cells. Particularly preferred plant cells include cells from monocotyledonous or dicotyledonous plants. Also preferred are cell lines developed from any of the foregoing types of cells.

Other features and advantages of the invention will be apparent from the following drawings, detailed description, and appended claims.

### BRIEF DESCRIPTION OF THE DRAWINGS

These and other aspects and embodiments of the present invention will become evident upon reference to the following detailed description and attached drawings that represent certain preferred embodiments of the invention, which drawings can be summarized as follows:

FIG. 1 is an illustration of a multi-well electroporation plate according to the invention. Depicted is a 96-well plate (2). Wells (4) are organized into eight rows (6) and twelve columns (8). The top surfaces (10) of each row are plated with a conductor to carry current. Extending into the wells are electrodes (12 and 14) connected to the conductors. Conductors for anodes are located on one side of a row, cathodes and their conductors on the other side. In this embodiment, electrical connection to an external energy source occurs at the edge of the plates, where electrical contacts with a power supply are made at the distal regions (16 and 18) of the connectors for anodes and cathodes.

FIG. 2 depicts various components of a representative electroporation system for multi-well electroporation plates according to the invention.

FIG. 3 depicts various representative embodiments of large volume electroporation plates according to the invention. FIG. 3A is an illustration of a large-scale electroporation plate (30) according to the invention, wherein the plate is suitable for large volume applications and comprises three identical electroporation chambers (32), each of which can hold up to about 15 mL of cell suspension. A shown, each of the electroporation chambers (32) are mounted on a base plate (30). Each chamber contains an inlet port (34) and an outlet port (38), as well as a vent (36) to facilitate filling and emptying of the chamber. Also, in the depicted embodiment, each chamber has an ergonomic portion (40) to facilitate handling.

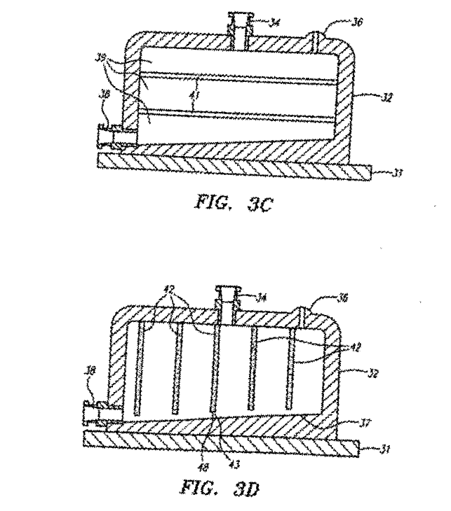

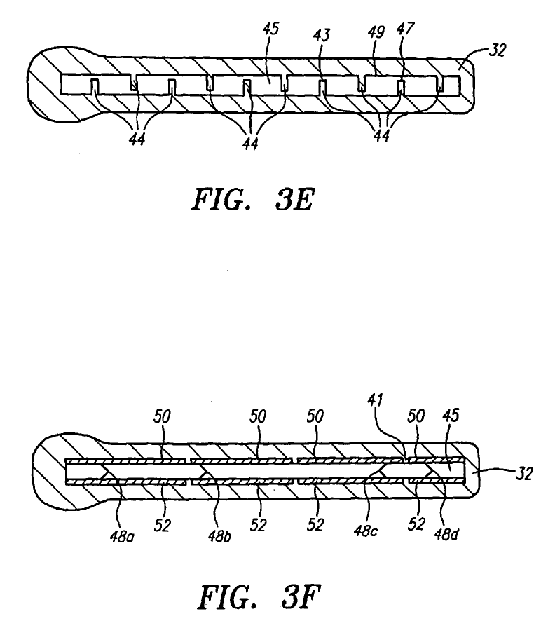

In the illustration shown in FIG. 3B, one of the chambers of the electroporation plate depicted in FIG. 3A is shown in a cutaway view to show a representative layout of electrodes. Here, three electrodes (39) disposed on one wall of the inside of chamber the (32) are represented, and each electrode on the wall is separated from another electrode by an insulating portion of the wall (41). In a chamber, each of these electrodes (39) would be paired with an electrode (39, not shown) on the opposite wall, and each electrode pair would be independently energizable. Preferably, the electrodes of each electrode pair are the same size and are disposed directly opposite one another. The electrodes of an electrode pair are also preferably spaced such that the surface of one electrode is parallel to the surface of the other electrode to ensure generation of electric fields of substantially equal strength between the electrodes, regardless of the position between the electrodes where field strength is measured. In the embodiment shown in FIG. 3B, the floor (37) of the solution-containing portion of the chamber (32) is tapered in the direction of the outlet port (38) to assist in draining a cell suspension from the chamber.

FIG. 3C shows a cross section of an alternative embodiment of the chambers (32) of FIG. 3A wherein the electrodes (39) are disposed substantially horizontally, as opposed to vertically. As in the embodiment of FIG. 3B, the electrodes (39) depicted on one of the inner walls of the chamber are separated from one another by a small insulating portion (41) of the wall.

FIGS. 3D and 3E illustrate embodiments wherein the chamber (32) has a series of internal baffles (42 in FIG. 3D, 44 in FIG. 3E) that optionally may be included in the device. When present, the baffles serve to limit the movement of liquid in the sample-containing portion (45) of the chamber (39). In the depicted embodiments, each chamber also has a sample inlet port (34) and a sample outlet port (38) to allow one way sample flow into and out of the chamber. FIG. 3D shows a cross-section taken through a chamber (32) to show a representative configuration of baffles (42). As depicted, each baffle extends downward from the top of the chamber, leaving an opening (43) between the bottom surface (37) of the chamber (32) and the lower face (48) of the baffle. Preferably, when multiple baffles are included in a chamber in this way, the opening below one baffle is the same size as the openings beneath the other baffles, although configurations having multiple baffles with differently sized openings are contemplated by the invention. As will be appreciated, the baffles may also contain one or more additional openings of various shapes and sizes over their length, and they may be attached to one or more sides of the chamber in addition to being attached to the top of the inside of the chamber.

FIG. 3E shows a related embodiment wherein the chamber (32) contains several baffles (44) extending from the side walls of the inside of the chamber in staggered fashion such that one baffle (44) leaves an opening (43) between its face (47) having the closet approach to the opposite wall face (49). As will be appreciated, any suitable embodiment containing one or more baffles, regardless of configuration, are contemplated by the invention. Indeed, the baffles may also contain electrodes (not shown).

FIG. 3F shows another representative embodiment of a chamber (32) for a large-scale electroporation plate (30) according to the invention that is suitable for large volume applications. In particular, FIG. 3F illustrates a top-down view of a single chamber of such a plate that comprises four electrode pairs (48a-d), with the anode (52) of each electrode pair being disposed directly opposite the cathode (50) of the pair. In the depicted embodiment, each electrode is integrated into and disposed vertically in a wall of the chamber directly opposite the other member of the particular electrode pair. On each of the walls that house the electrodes, the electrodes are separated from one another by an insulating portion (41) of the particular wall. The plate and chambers also comprise conductors (not shown) that connect each of the anodes and cathodes in a particular electrode set to electrical contacts that can be connected to a power supply (not shown) for energizing the electrodes. In the depicted embodiment, each electrode set (here, an electrode pair) may be energized independently of the other electrode sets.

As those in the art will appreciate, the embodiments represented in the attached drawings are representative only and do not depict the actual scope of the invention. For example, the various components of an electroplate plate according to the invention may be arranged differently or include additional and/or different components.

### DETAILED DESCRIPTION OF THE INVENTION

Before the present invention is described in detail, it is understood that the invention is not limited to the particular electroporation plates, systems, and methodologies described, as these may vary. It is also to be understood that the terminology used herein is for the purpose of describing particular embodiments only, and is not intended to limit the scope of the invention defined by the appended claims.

1. Introduction.

At its core, the present invention relates to large-scale electroporation plates. In certain preferred embodiments, these plates have at least two wells, and as many as 96, 192, 288, 384, 576, 672, 768, 1536, 3072, 6144, or more identical wells, that contain electroporation electrodes that can be independently energized or addressed. In other preferred embodiments, an electroporation plate according to the invention comprises at least one, and as many as 2-30 or more, large volume chambers for electroporating cell-containing suspensions. In such embodiments, each chamber comprises a plurality of electrode sets that can be independently energized or addressed.

It is understood that, in the context of this invention, “independently energized”, “independently addressed”, or the like refers to the capability of a pair of electroporation electrodes (or larger number of electrodes comprising an electrode set, e.g. opposed pairs of electrodes) to receive energy separate and apart from any other pair of electroporation electrodes in the plate. This capability is a function of the plate design, and can be accomplished many ways. For example, each electroporation electrode pair may be separately wired from the other electrode pairs, where each electrode pair is part of its own separate circuit comprised of the electrodes and the conductors that connect the electrodes to the electrical contacts for connection to a power supply. Alternatively, a plurality of electrodes, preferably electrode pairs, can be configured as elements of the same circuit, with each electrode pair having a switch associated with it. Thus, a switch will determine whether a particular electrode pair is energized when the circuit is energized. Of course, plates having combinations of such circuits can also be made. When the electrode pair (or set) for single well (or chamber) can be independently addressed, the well (or its electrodes) is (are) said to be “independently addressable”. Likewise, in other embodiments, where the plate is designed so that several, but not all, electrode pairs or sets are part of one circuit, and thus can be energized together (i.e., at the same time, without switch actuation), those electrode pairs (and their corresponding wells) are said to be independently addressable, as compared to other electrodes (and wells) in the plate.

In addition to electroporation plates and kits comprised of such plates loaded with electrically competent host cells, the invention also concerns electroporation systems that use such plates. At a minimum, Such an electroporation system will comprise a plate handler that energizes the circuit(s) present in an electroporation plate. Thus, a plate handler will comprise a power supply to provide the energy needed to energize a plate's electrodes, as well as a controller that directs when and how each circuit will be energized (and, if present, which switch or switches will be opened or closed). The controller and power supply may be separate units, although preferably these functionalities reside within a single device. An electroporation system can also include one or more plate readers, data storage devices, and/or computers (e.g., to optimize electroporation parameters, manage plate movement and handling, etc.).

Preferred embodiments of these and other aspects of the invention are thoroughly described below.

2. Electroporation Plates.

This invention concerns electroporation plates, articles of manufacture useful in performing electroporation experiments. With regard to embodiments useful for high-throughput applications, the plates each comprise a plurality of electrifiable wells, at least two of which can be independently energized. The wells are disposed in a solid substrate, and may be introduced at various stages of manufacture. To electrify each well, a at least two electrodes (an anode and a cathode) are permanently disposed therein. As with the wells themselves, the electrodes may be introduced at various stages of manufacture. The electrodes are energized using an external power supply (often referred to in the electroporation field as a “pulse generator”) that can be operatively coupled to the plate. Similarly, in embodiments where the plates comprise one or more large volume chambers, each chamber comprises a plurality of electrode sets that can be independently energized. Each of the electrodes in an electrode set is preferably disposed in a wall of the chamber, which is typically made from a solid substrate. As will be appreciated, chambers can be made separately and then be attached to a support member at an appropriate stage. Accordingly, the electrical circuitry for connecting the electrode sets to a power supply can be integrated solely in the body of the chamber itself or, alternatively, it can include components some of which reside in the chamber and some of which reside on the support. In other embodiments, the chambers and support are manufactured as an integrated unit, in which event the completed unit will comprise the conductors and electrical connections necessary for the electrodes of the device to be energized by a power supply.

a. Design.

As described herein, a high throughput electroporation plate according to the invention comprises a plurality of electrifiable wells, at least two of which can be independently energized. Because such plates are likely to be used in conjunction with automated, high throughput screening systems, it is preferred that the plates be designed to operate in such an environment, although such compatibility is not essential. Developers of hardware for most high throughput biomolecular screening systems rely on standards published by the Society for Biomolecular Screening (Danbury, Conn.) and approved by the American National Standards Institute, Inc. Among other things, such standards (be they draft, interim, or formally adopted) have defined a set of common dimensions for plates to be used in these systems. Generally, standards exist for plate footprint dimensions, height dimensions, bottom outside flange dimensions, well positions (for example, for 96, 384, and 1536 well plates), and side-wall rigidity. Plates of these dimensions may be readily handled by robotic systems and automated hardware platforms in use in many pharmaceutical, biotechnology, and agricultural biosciences corporations, as well as academic and other research institutions throughout the world. To facilitate widespread adoption of the instant electroporation plates for high throughput applications, it is desirable that the plates conform to these or later developed standards to support automation and cross-platform compatibility.

Preferred examples of such embodiments include those wherein an electroporation plate comprises a plurality of wells organized in columns and rows. In some of these embodiments, two or more of the wells in a given column (or row)(which may or may not be all of the wells in the column) are energized together, but independently of the wells in other columns (or rows) of the plate. In others, the electrodes of all of the wells of a given column or row will be energized together, but independently from, the other electrodes of the plate. In such embodiments, it is preferred that the electrodes of a particular column (or set of columns or portions thereof) to be energized together be operably connected such that when one electrode pair is energized, all of them are energized with energy having the same energizing parameters, A “portion of” the wells (or electrodes) of a column or row refers to two or more, but fewer than all, of the wells (or electrodes) of the column or row.

In other embodiments, the electroporation plates of the invention comprise one or more large volume chambers for conducting large-scale electroporation. In such embodiments, each chamber comprises a plurality of electrifiable electrode sets that each minimally comprise at least two electrodes. Each of the electrode sets can be independently energized. As will be appreciated, such plates can be designed such that the plate and chamber(s) are manufactured as a single part. Alternatively, the plate may be designed to comprise two or more pieces that can be assembled as needed or desired. For example, the plate may comprise a support member manufactured to receive one or more large volume chambers. In such embodiments, the chambers may be of the same or different size, i.e., each chamber may have the capacity to hold the same or a different volume of cells. In multi-piece assemblies, the support member may or may not contain electrical components. In embodiments where the support member does not have electrical components, the chamber units comprise the necessary circuitry and connectors to allow the electrodes to be energized after connection to a suitable power supply (i.e., an electroporation pulse generator). In embodiments where the support member does comprise certain of the components necessary to energize the electrodes, the support member and chamber(s) comprise such connectors or other components as are necessary to make the desired connections between the electrodes and a suitable power supply. Of course, the invention contemplates a plethora of potential configurations for the plates of the invention. As such, the exact design of a plate according to the invention is left to the discretion of the skilled artisan.

b. Solid Substrates.

Electroporation plates according to the invention can be made from any suitable solid substrate. Preferred substrates are those that can be manufactured to the desired specifications by molding processes and/or machining. Particularly preferred are plastics or other polymers that can be formed into the desired shape by injection molding or similar processes. Polycarbonate, acrylonitrile butadiene styrene (ABS), and polystyrene are particularly preferred this reason, although any material (or combinations of different materials) that can be fashioned into a large-scale electroporation according to the invention may be employed. In embodiments that employ plastics, it will be appreciated that the plastics may be impregnated and/or reinforced with materials to provide desired characteristics, e.g., improved rigidity, heat dissipation, insulation against electric current flow, etc.

Another advantage of plastics is the ability to use different colors and differing degrees of transparency in different parts of an electroporation plate according to the invention. For example, many detection systems are based on detecting light of specific wavelengths. For this reason, in many embodiments, the bottom of an electroporation plate is made from a transparent plastic, while portions of the plate that form the sides of the wells are made from an opaque plastic.

While plastics represent preferred examples of solid substrates for forming an electroporation plate according to the invention, other embodiments employ ceramics or metals. As these materials, and techniques for their preparation, are well known in the art, such materials may be readily adapted for use in practicing the invention by those ordinarily skilled in the art in light of this specification.

c. Electrodes.

Electrodes can be made or formed from any suitable electrically conductive material, or combination of materials, including composites of such materials. Preferably, the material(s) used for electrodes will be biocompatible. When an electrode is formed from multiple materials (e.g., an electrically conductive material such as gold plated over an electrically conductive material such as nickel plated over an electrically conductive material such as copper, or a carrier doped with a electrically conductive material), it is preferred that at least the outermost layer, i.e., the layer that will be exposed to a cell suspension, is biocompatible. Preferred biocompatible electrode materials include gold and titanium. Of course, other conductors may also be used (e.g., aluminum, various stainless steels, etc.), and their selection is left to the discretion of the skilled artisan based on the particular application.

Electrodes and conductors can be included in an electroporation plate by any suitable process. For example, in some embodiments, electrode materials are plated or otherwise deposited onto the solid substrate in desired locations. In other embodiments, the solid substrate is machined to accept electrodes for one or more pre-formed wells.

d. Manufacture.

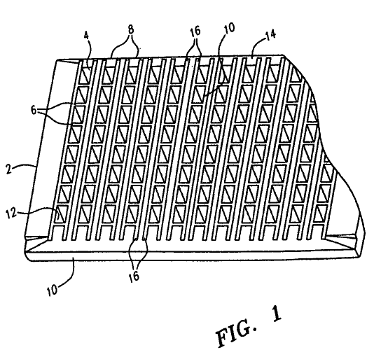

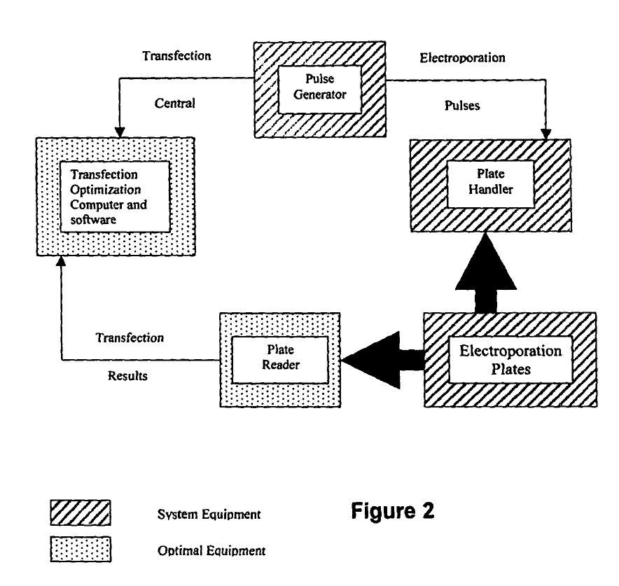

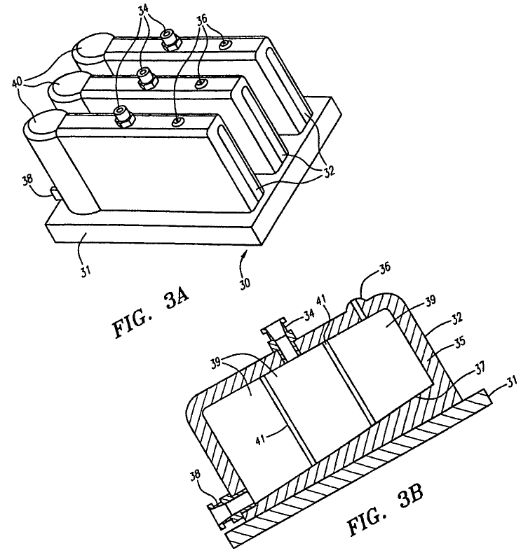

A particularly preferred embodiment of a high throughput electroporation plate according to the invention (see FIG. 1) has been produced by a multi-step process, as follows. A polycarbonate frame providing the basic structure for a 96-well electroporation plate was formed by injection molding. The 96 wells were arranged in eight rows and twelve columns. The side walls of each well disposed in the direction of its row, as well as the top of the row adjacent to each well, was then molded with a material such as ABS or polycarbonate/ABS (PC-ABS) plastic blend to provide a plate having surface properties on certain of its surfaces (here, the well side walls on the row sides of the well and tops of the rows) suitable for plating metals or other materials useful as electrodes and conductors. After the wells were molded, the plate was exposed to an etching solution to prepare the surfaces for application (e.g., by plating) of the electrode composition.

In this electroporation plate, three different layers of metal were sequentially deposited as relatively thin films on certain portions the solid substrate (e.g., wells formed from ABS over a polycarbonate frame) using plating techniques that would differentiate between the polycarbonate and ABS materials. Initially, a relatively thin copper film of a nominal thickness of about 10 microinches (mi) was plated onto the ABS portions of the plate using electroless copper plating techniques. Copper was chosen in this case because it adheres well to ABS and provides transition bonding between the plastic and metal layers. Next, a relatively thick film of copper having a nominal thickness of about 1/1000 inch (1 mil) was deposited on the initial copper layer using electroplating techniques. In plates such as these, these copper layers provide the bulk of current carrying capacity. After the copper was applied, a thin film of nickel having a nominal thickness of about 100 ml was deposited on the copper. Nickel was used because of its conductive properties and because it adheres well to the copper layer and forms a good substrate for electroplating gold. Finally, a finish layer of gold was plated on the nickel. Because of its relative cost, the gold was electrically plated using a controlled electroplating technique. The gold layer had a nominal thickness of about 10 mi. As will be appreciated, due to cost considerations, the nominal thickness of the copper and nickel layers can vary as much as 50% or more. The thickness of the gold layer was more tightly controlled, and preferably varies less than about 5%. Electrodes useful in practicing the invention, including multi-layer electrodes such as those described above, preferably have the capacity for use under a variety of different electroporation conditions (including 400 volts (V) for 10 milliseconds (msec) using a standard buffered saline solution), making a particular plate useful for a variety of different electroporation conditions. Indeed, given that some or all of the wells can be independently electrically addressed (i.e., energized) as compared to other wells on the plate, a single plate can be used to test a plurality of different electroporation conditions. For purposes of quality control, the electrodes

Such three-layer electrodes as described above were calculated to have nominal track resistance of 0.18 ohm for the copper layer, 0.73 ohm for the nickel layer, and 2.5 ohm for the gold layer, yielding a total resistance of about 0.136 ohm. It is believed that about 75% of an applied electric current (i.e., an electroporation pulse) will flow through the copper, although it will migrate into the nickel and then the gold layer to reach the sample.

After adding the electrode layers, a transparent, insulating bottom made of plastic (e.g., polyester or polystyrene) was bonded to the other portions of plate using an adhesive cured under ultraviolet light so as to ensure that the bottom of each well was completely sealed to prevent well-to-well leakage. The plate bottom may be clear, translucent, or opaque, as may other parts of the plates. It has been found that when the plates are to be used in luminescence-based assays, white bottoms are preferred. In any event, after a suitable bottom has been affixed to the plate, the plate preferably is then sealed and sterilized.

In another particularly preferred embodiment of a high throughput electroporation plate according to the invention, an insulating polymer is over-molded onto an array of electrically conductive electrodes configured so as to ultimately provide a plate having 96 wells arranged in an 8×12 array such that each well contains at least a pair of electrodes, and wherein the electrodes of some or all of the wells may be energized independently of the electrodes of other wells in the plate.

3. Applications.

Because the high throughput electroporation plates of the invention provide at least two wells that contain electroporation electrodes that can be independently energized or addressed, multiple electroporation experiments can be performed in a single plate. Similarly, because certain large-scale electroporation plates according to the invention comprise one or more large volume electroporation chambers, each of which contain two or more electrode sets that can be independently energized or addressed, large volumes of cells can be efficiently electroporated in a single vessel. Moreover, existing electroporation power supplies can be used more efficiently when used in conjunction with such plates, as it is now possible to energize different regions of a plate, or different electrode sets of a single chamber, at different times, thereby allowing the power supply to recharge between energizings. Thus, electroporation conditions can be readily optimized using conventional pulse generators.

Another application of the invention involves introducing exogenous chemicals such as nucleic acid molecules into host organisms (such as eukaryotic and prokaryotic host cells), as such techniques are central to many types of experiments, analyses, and therapies. For example, when searching for a gene of interest in a DNA library, the library must first be introduced into a suitable host cell population. Since a typical DNA library (e.g., a library for the genome of organism such man, mouse, corn, etc.) is very complex (i.e., contains thousands, tens of thousands, or more different genes), the number of independent clones needed to completely represent each gene in the library is large. To create a library that completely represents an organism's genome, for example, the efficiency at which DNA can be introduced into the host cells may become limiting. By optimizing this process, the ability to create and screen DNA libraries is facilitated.

Similarly, many other experimental analyses are limited by the ability to introduce DNA into a host organism. When cloning large segments of DNA for whole genome analysis (i.e., using bacterial artificial chromosomes), when performing cloning using the polymerase chain reaction (PCR), or when carrying out random mutagenesis of a gene, followed by cloning all potential altered forms, success often depends on the size of the initial transformation pool. Again, developing conditions that improve the process of introducing nucleic acids into a host organism increases the chance that the experiment will succeed.

In addition to experimental uses, electroporation can be used for therapeutic purposes. Here, the plates of the invention can be used to introduce exogenous molecules having a therapeutic benefit into cells of a patient, particularly in ex vivo formats. Examples of therapeutic exogenous molecules include nucleic acid molecules. Nucleic acids can be delivered to effect so-called “gene therapy”, i.e., the introduction into a patient of one or more genes intended to produce a therapeutic benefit. Alternatively, the large-scale electroporation plates of the invention can be used in an ex vivo format to introduce drugs from other drug classes (e.g., small molecule pharmaceuticals, therapeutic proteins, etc.) into cells of. After treatment, the cells may then be introduced into a patient. Often, the cells will be reintroduced into the patient from whom they were removed.

In developing and refining electroporation methodology, factors have been identified that impact the efficiency of the transfer of exogenous molecules, e.g., nucleic acids. These factors include electrical field strength, pulse decay time, pulse shape, reaction temperature, cell type, suspension buffer composition, and the concentration and size of the species (including more than one species) of exogenous molecule, e.g., a nucleic acid molecule, to be transferred. Given the number of parameters that can influence the efficiency of an electroporation experiment, in research and commercial settings it is often important to define conditions that will result in high efficiency transfer of the desired molecules (e.g., recombinant nucleic acids) into a particular host cell line. Thus, optimization will be important to increasing the use and reproducibility of electroporation in biomolecular studies.

To perform optimization, an electroporation system of the invention will typically further comprise an optimization computer adapted to optimize electroporation conditions, alone or in conjunction with other experimental conditions, using electroporation data stored in the memory. Because a high throughput electroporation plate according to the invention comprises a plurality of independently addressable wells, a plurality of electroporation experiments can be performed. Here, an “electroporation experiment” refers to the particular set of energizing parameters used to energize the electrodes in a given well (preferably, several wells). Thus, a plurality of different electroporation experiments can be performed on a single plate. Analysis of the resulting data enables electroporation conditions (i.e., the energizing parameters and other conditions, e.g., buffer, cell concentration, etc.) to be optimized for a particular cell line or cell population. Optimization is preferably performed using statistical methods and techniques such as multi-variate analysis so that optimal electroporation conditions (or at least energizing parameters for the given host cell, buffer, and exogenous molecule) can be determined.

All patents and patent applications, publications, scientific articles, and other referenced materials mentioned in this specification are indicative of the levels of skill of those skilled in the art to which the invention pertains, and each of which is hereby incorporated by reference to the same extent as if each reference had been incorporated by reference in its entirety individually. Applicants reserve the right to physically incorporate into this specification any and all materials and information from any such patents and patent applications, publications, scientific articles, electronically available information, and other referenced materials or documents.

The specific electroporation plates, systems, and methods described in this specification are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Also, the terms “comprising”, “including”, “containing”, etc. are to be read expansively and without limitation. It must be noted that as used herein and in the appended claims, the singular forms “a”, “an”, and “the” include plural reference unless the context clearly dictates otherwise.

The terms and expressions that have been employed are used as terms of description and not of limitation, and there is no intent in the use of such terms and expressions to exclude any now-existing or later-developed equivalent of the features shown and described or portions thereof, but it is recognized that various modifications are possible within the scope of the invention as claimed. Thus, it will be understood that although the present invention has been specifically disclosed by preferred embodiments and optional features, modification and/or variation of the disclosed elements may be resorted to by those skilled in the art, and that such modifications and variations are within the scope of the invention as claimed.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. An electroporation plate, comprising:
a. a plurality of energizable electroporation wells arrayed in a solid substrate, wherein each electroporation well comprises at least two electroporation electrodes disposed therein, and wherein the at least two electroporation electrodes in at least two of the wells can be independently energized; and
b. electrical connections for connecting the electroporation electrodes to an energy source.

2. An electroporation plate according to claim 1 that comprises about 2, 12, 24, 96, 192, 288, 384, 576, 768, 672, 1536, 3072, or 6144 electroporation wells.

3. An electroporation plate according to claim 1 wherein the electroporation wells are of substantially uniform dimensions.

4. An electroporation plate according to claim 1 wherein the electroporation wells are substantially either cylindrical or rectangular.

5. An electroporation plate according to claim 1 wherein each electroporation well has a volume between about 1 uL and about 10 mL.

6. An electroporation plate according to claim 1 wherein the electroporation wells have a volume of about 1 uL to about 1 mL.

7. An electroporation plate according to claim 1 wherein the electroporation wells have a volume of about 1 mL to about 10 mL.

8. An electroporation plate according to claim 1 wherein one of the electroporation electrodes in an electroporation well is disposed opposite of the other electroporation electrode.

9. An electroporation plate according to claim 1 wherein each electroporation well comprises at least one sidewall and a bottom wall.

10. An electroporation plate according to claim 9 wherein the electroporation electrodes of an electroporation well are integrated into the sidewall.

11. An electroporation plate according to claim 1 wherein the electroporation wells arranged in a plurality of rows and columns.

12. An electroporation plate according to claim 11 wherein the electroporation electrodes in the electroporation wells of a row are operably connected so as to be simultaneously energized.

13. An electroporation plate according to claim 12 wherein the operably connected electroporation electrodes in the electroporation wells of one row can be energized independently of the electroporation electrodes in electroporation wells in other rows of the electroporation plate.

14. An electroporation plate according to claim 13 wherein the electroporation electrodes in each row are energized independently of the electroporation electrodes in other rows of the electroporation plate.

15. An electroporation plate according to claim 1 wherein the electrodes in each well comprise a plurality of electrodes that are each individually energizable.

16. An electroporation plate according to claim 15 wherein said plurality of electrodes comprises between 2 and 12 pairs of electrodes.

17. An electroporation plate according to claim 16 wherein the electrodes of each pair of electrodes comprise a cathode and an anode positioned in said well opposite to one another.

18. An electroporation plate according to claim 17 wherein at least two adjacent pairs of electrodes are energized simultaneously as an electrode set.

19. An electroporation plate according to claim 1 wherein the electroporation electrodes in each electroporation well are independently energizable.

20. An electroporation plate according to claim 1 wherein the material comprising the solid substrate is selected from the group consisting of plastic, metal, and ceramic.

21. An electroporation plate according to claim 1 wherein the solid substrate is transparent.

22. An electroporation plate according to claim 1 wherein the solid substrate is translucent.

23. An electroporation plate according to claim 1 wherein the solid substrate is opaque.

24. An electroporation plate according to claim 1 wherein the electroporation electrodes are integrated in the solid substrate.

25. An electroporation plate according to claim 1 wherein the electroporation electrodes are deposited on a surface of the solid substrate.

26. An electroporation plate according to claim 1 wherein the electroporation electrodes are deposited by vapor deposition.

27. An electroporation plate according to claim 26 wherein the electroporation electrodes comprise an electrically conductive material or a combination of electrically conductive materials.

28. An electroporation plate according to claim 26 wherein the electrical connection comprises pins and sockets.

29. An electroporation plate according to claim 26 wherein the electrical connection comprises an independent electrical contact for each pair of electroporation electrodes that is independently energized.

30. An electroporation system, comprising:
a. an electroporation plate according to claim 1; and
b. a power supply adapted for connection to the electrical connector of the electroporation plate.

31. An electroporation system according to claim 30 further comprising a plate handler configured to hold the electroporation plate during operation of the electroporation system.

32. An electroporation system according to claim 30 further comprising a plate reader.

33. An electroporation system according to claim 32 wherein the plate reader uses a reading element selected from the group consisting of a machine vision device, a spectrophotometer, and a luminomitor to collect electroporation data by examining the contents of one or more of the electroporation wells of the electroporation plate.

34. An electroporation system according to claim 32 wherein the plate reader is integrated into the plate handler.

35. An electroporation system according to claim 30 wherein the plate handler is a robotic plate handler.

36. An electroporation system according to claim 35 further comprising a plate storage facility for storing electroporation plates during the course or following completion of an electroporation experiment.

37. An electroporation system according to claim 36 wherein the plate storage facility is an incubator configured to hold a plurality of electroporation plates.

38. An electroporation system according to claim 33 further comprising a data storage device to store data collected by the plate reader.

39. An electroporation system according to claim 38 further comprising an optimization computer adapted to optimize electroporation conditions from electroporation data stored in the memory.

40. A method of introducing an exogenous molecule into a host cell, comprising using electroporation to introduce the exogenous molecule into the host cell in a suspension contained in an electroporation well of an electroporation plate according to claim 1.

41. A method according to claim 40 wherein the exogenous molecule is a nucleic acid.

42. A method according to claim 40 wherein the host cell is selected from the group consisting of eukaryotic cell and a prokaryotic cell.

43. A method according to claim 42 wherein the host cell is a eukaryotic cell selected from the group consisting of an animal cell and a plant cell.

44. A method according to claim 43 wherein the eukaryotic cell is an animal cell selected from the group consisting of an mammalian cell, an insect cell, a fish cell, a bird cell, an arachnid cell, a mollusk cell, and a crustacean cell.

45. A method according to claim 44 wherein the eukaryotic cell is a mammalian cell from a mammal selected from the group consisting of bovine, canine, equine, feline, murine, ovine, and porcine animals.

46. A method according to claim 43 wherein the eukaryotic cell is a cell from a mammalian cell line.

47. A method according to claim 43 wherein the eukaryotic cell is a plant cell from a monocotyledonous plant or a dicotyledonous plant.
